(19) United States
(12) Patent Application Publication
(10) Pub. No.: US 2024/0350665 A1
(43) Pub. Date: Oct. 24, 2024

(54) HEPATITIS B VIRUS (HBV) KNOCKOUTS

(71) Applicant: EmendoBio Inc., Wilmington, DE (US)

(72) Inventor: Rafi EMMANUEL, Ramla (IL)

(73) Assignee: EmendoBio Inc., Wilmington, DE (US)

(21) Appl. No.: 18/291,051

(22) PCT Filed: Jul. 21, 2022
(86) PCT No.: PCT/US22/73981

§ 371 Date: Jan. 22, 2024

Related U.S. Application Data
(60) Provisional application No. 63/224,581, filed on Jul. 22, 2021.

(51) Int. Cl. A61K 48/00; C12N 7/00; C12N 9/22; C12N 15/11

(52) U.S. Cl. CPC A61K 48/005; C12N 7/00; C12N 9/22; C12N 15/111; C12N 2310/20; C12N 2730/10121; C12N 2730/10162

(57) ABSTRACT

RNA molecules comprising a guide sequence portion having 17-50 contiguous nucleotides containing nucleotides in the sequence set forth in any one of SEQ ID NOs: 1-18936 and compositions, methods, and uses thereof.

This application claims the benefit of U.S. Provisional Application 63/224,581, filed Jul. 22, 2021, the content of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “220721_91770-A-PCT_Sequence_Listing_AWG.xml”, which is 23,850 kilobytes in size, and which was created on Jul. 19, 2022 in the IBM-PC machine format, having an operating system compatibility with MS-Windows, which is contained in the XML file filed Jul. 21, 2022 as part of this application.

### BACKGROUND OF INVENTION

### Hepatitis B Virus and Liver Infection

Hepatitis B virus (HBV) is the causative agent of hepatitis B liver infection (which is also referred to as “hepatitis B”). According to the CDC, while hepatitis B is a short-term illness for many, it can become a long-term, chronic infection that can lead to serious health issues like cirrhosis or liver cancer. Risk for chronic infection is related to age at infection: about 90% of infants with hepatitis B go on to develop chronic infection, whereas only 2%-6% of people who get hepatitis B as adults become chronically infected. An efficient cure of chronic HBV infection will require elimination of the HBV covalently closed circular DNA (cccDNA), which is the long-lived viral genomic intermediate that is the template for HBV replication and persistence.

### SUMMARY OF THE INVENTION

Disclosed is an approach for disrupting a HBV genomic DNA molecule in a cell. According to some aspects of the invention, the approach results in knocking out the expression of an HBV gene. In some embodiments, the present disclosure provides a method for disrupting a conserved region of an HBV sequence or a portion thereof. In some embodiments, the present disclosure provides a method for disrupting a regulatory element of an HBV sequence or a portion thereof. In some embodiments, the present disclosure provides a method for disrupting a coding sequence of an HBV sequence or a portion thereof. In some embodiments, an HBV sequence is targeted and modified within an HBV covalently closed circular DNA (cccDNA) molecule and/or within a mammalian host genomic DNA molecule. According to some aspects, the present disclosure provides a method for targeting and modifying a sequence of an HBV covalently closed circular DNA (cccDNA) molecule.

The present disclosure also provides a method for modifying a hepatitis B virus (HBV) gene in a cell containing HBV, the method comprising
introducing to the cell a composition comprising:
   at least one CRISPR nuclease or a nucleotide sequence encoding a CRISPR nuclease; and
   a first RNA molecule comprising a guide sequence portion having 17-50 nucleotides or a nucleotide sequence encoding the same,
wherein a complex of the CRISPR nuclease and the first RNA molecule affects a double strand break in the HBV gene.

According to embodiments of the present invention, there is provided a first RNA molecule comprising a guide sequence portion having 17-50 contiguous nucleotides containing nucleotides in the sequence set forth in any one of SEQ ID Nos: 1-18936.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-18936 and a CRISPR nuclease.

According to some embodiments of the present invention, there is provided a method for treating hepatitis B, the method comprising delivering to a cell of a subject having hepatitis B a composition comprising an RNA molecule comprising a guide sequence portion having 17-50 contiguous nucleotides containing nucleotides in the sequence set forth in any one of SEQ ID NOs: 1-18936 and a CRISPR nuclease.

According to some embodiments of the present invention, there is provided a method for inactivating a hepatitis B virus in a cell, the method comprising delivering to a cell containing hepatitis B virus a composition comprising an RNA molecule comprising a guide sequence portion having 17-50 contiguous nucleotides containing nucleotides in the sequence set forth in any one of SEQ ID NOs: 1-18936 and a CRISPR nuclease.

### BRIEF DESCRIPTION OF THE DRAWINGS

FIG. 1A shows a schematic representation of HBV cccDNA (linearized) molecule, and FIG. 1B shows how HBV segments were cloned into a lentiviral vector for use in subsequent infection of HeLa cells.

FIG. 2 shows activity of guide molecules targeting HBV in HeLa cells. Specific guide molecules were-co-transfected with wild-type OMNI-79 (WT) nuclease or the OMNI-79 V5570 variant nuclease to determine the on-target activity of the guide molecules. Cells were harvested 72 hours post DNA transfection, genomic was DNA extracted, the region containing the nuclease cut-site was amplified, and then analyzed by next-generation sequencing (NGS). The graph represents the % of editing±STDV of three independent transfections in cells transfected with lentivirus at a multiplicity of infection (MOI) of 2.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions) whereas during HDR, an intact homologous DNA donor is used to replace the DNA surrounding the cleavage site in an accurate manner. HDR can also mediate the precise insertion of exogenous DNA at the break site. Accordingly, the term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR molecule, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule, the RNA molecule is capable of targeting the CRISPR nuclease to the specific target DNA sequence. Each possibility represents a separate embodiment. An RNA molecule can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs:1-18936. Each possibility represents a separate embodiment. In some of these embodiments, the guide sequence portion is fully complementary to the target sequence and comprises a sequence that is the same as a sequence set forth in any of SEQ ID NOs:1-18936. Throughout this application, the terms “guide molecule,” “RNA guide molecule,” “guide RNA molecule,” and “gRNA molecule” are synonymous with a molecule comprising a guide sequence portion, and the term “spacer” is synonymous with a “guide sequence portion.”

In embodiments of the present invention, an RNA molecule comprises a guide sequence portion having 17-50 contiguous nucleotides containing nucleotides in the sequence set forth in any one of SEQ ID NOs: 1-18936.

The RNA molecule and or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-18936. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-18936. For example, a guide sequence portion having 17 nucleotides in the sequence of 17 contiguous nucleotides set forth in SEQ ID NO: 18937 may consist of any one of the following nucleotide sequences (nucleotides excluded from the contiguous sequence are marked in strike-through):

(SEQ ID NO: 18937)
AAAAAAAUGUACUUGGUUCC
17 nucleotide guide sequence 1:
(SEQ ID NO: 18938) [symbol]
AAAAUGUACUUGGUUCC
17 nucleotide guide sequence 2:
(SEQ ID NO: 18939) [symbol]
AAAAAUGUACUUGGUUC [symbol]
17 nucleotide guide sequence 3:
(SEQ ID NO: 18940) [symbol]
AAAAAAUGUACUUGGUU [symbol]
17 nucleotide guide sequence 4:
(SEQ ID NO: 18941)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24 or 25 nucleotides in length. In such embodiments the guide sequence portion comprises 17-50 nucleotides containing the sequence of 20, 21 or 22 contiguous nucleotides set forth in any one of SEQ ID NOs: 1-18936 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

The term “conserved region” as used herein refers to a region of a nucleotide molecule or of an amino acid molecule having sequence identity across several different species or strains. Similarly, the term “conserved sequence” as used here refers to a sequence having sequence identity across several different species or strains. For example, a conserved sequence of HBV DNA is an HBV DNA sequence that has sequence identity across several different strains, variants, or serotypes of HBV. The sequence identity may be, for example: at least 70% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, or 90% sequence identity across several HBV strains, variants, or serotypes.

According to embodiments of the present invention, there is provided a method for modifying a hepatitis B virus (HBV) sequence in a cell, the method comprising
introducing to the cell a composition comprising:
   at least one CRISPR nuclease or a nucleotide sequence encoding a CRISPR nuclease; and
   a first RNA molecule comprising a guide sequence portion having 17-50 nucleotides or a nucleotide sequence encoding the same,
wherein a complex of the CRISPR nuclease and the first RNA molecule affects a double strand break in the HBV sequence.

In some embodiments, the guide sequence portion of the first RNA molecule comprises 17-50 contiguous nucleotides containing nucleotides in the sequence set forth in any one of SEQ ID NOs: 1-18936.

In some embodiments, a double-strand break is affected in a hepatitis B virus DNA sequence. In some embodiments, a double-strand break is affected in an HBV gene or portion thereof, an HBV coding sequence or portion thereof, an HBV regulatory sequence or portion thereof, and/or a conserved HBV sequence.

In some embodiments, a double-strand break is affected up to 500 nucleotides upstream or downstream to an HBV coding sequence, an HBV regulatory sequence, and/or a conserved HBV sequence. Each possibility represents a separate embodiment. In some embodiments, an HBV conserved region is targeted.

In some embodiments, the method further comprises introducing to the cell a second RNA molecule comprising a guide sequence portion having 17-50 nucleotides or a nucleotide sequence encoding the same, wherein a complex of the second RNA molecule and a CRISPR nuclease affects a second double strand break in a hepatitis B virus sequence.

In some embodiments, the guide sequence portion of the second RNA molecule comprises 17-50 contiguous nucleotides containing nucleotides in the sequence set forth in any one of SEQ ID NOs: 1-18936 other than the sequence of the first RNA molecule.

In some embodiments, a sequence of nucleotides is excised from a molecule comprising the HBV sequence.

In some embodiments, the sequence of nucleotides excised from the HBV sequence comprises an HBV gene or portion thereof, an HBV coding sequence or portion thereof, an HBV regulatory sequence or portion thereof, and/or a conserved HBV sequence.

In some embodiments, an HBV gene or a portion thereof is excised. In some embodiments, an HBV regulatory sequence or a portion thereof is excised. In some embodiments, an HBV non-coding sequence or a portion thereof is excised.

In some embodiments, the first or second RNA molecule comprises a guide sequence portion that targets a sequence that is located up to 500 base pairs from an HBV gene, an HBV coding sequence, an HBV regulatory sequence, and/or a conserved HBV sequence that is to be excised by the first and second RNA molecules.

In some embodiments, the HBV sequence is excised from an HBV cccDNA molecule.

In some embodiments, the HBV sequence is excised from a genomic DNA molecule that an HBV sequence has integrated into.

In some embodiments, the cell is a liver cell or a hepatocyte.

In some embodiments, the cell is in a human subject.

In some embodiments, the human subject suffers from chronic or acute hepatitis B.

In some embodiments, the HBV sequence is located on an HBV covalently closed circular DNA (cccDNA) molecule.

In some embodiments, the HBV sequence is located on a genomic DNA molecule that an HBV sequence has integrated into.

According to embodiments of the present invention, there is provided a composition comprising a first RNA molecule, the first RNA molecule comprising a guide sequence portion having 17-50 contiguous nucleotides containing nucleotides in the sequence set forth in any one of SEQ ID NOs: 1-18936.

In some embodiments, the composition further comprises at least one CRISPR nuclease.

In some embodiments, the composition further comprises a second RNA molecule comprising a guide sequence portion having 17-50 contiguous nucleotides, wherein the second RNA molecule targets a HBV gene, and wherein the guide sequence portion of the second RNA molecule is a different sequence from the sequence of the guide sequence portion of the first RNA molecule.

In some embodiments, the guide sequence portion of the second RNA molecule comprises 17-50 contiguous nucleotides containing nucleotides in the sequence set forth in any one of SEQ ID NOs: 1-18936 other than the sequence of the first RNA molecule.

According to embodiments of the present invention, there is provided a method for inactivating a hepatitis B virus in a cell, the method comprising delivering to the cell the composition of any one of the above embodiments.

According to embodiments of the present invention, there is provided a method for treating hepatitis B comprising delivering to a cell of a subject having hepatitis B the composition of any one of the above embodiments.

According to embodiments of the present invention, there is provided a use of the composition any one of the above embodiments for inactivating a hepatitis B virus in a cell, comprising delivering to the cell the composition.

According to embodiments of the present invention, there is provided a medicament comprising the composition of any one of the above embodiments for use in inactivating a hepatitis B virus in a cell, wherein the medicament is administered by delivering to the cell the composition.

According to embodiments of the present invention, there is provided a there is provided a use of the composition any one of the above embodiments for treating ameliorating or preventing hepatitis B, comprising delivering to a cell of a subject having or at risk of having hepatitis B the composition.

According to embodiments of the present invention, there is provided a medicament comprising the composition of any one of the above embodiments for use in treating ameliorating or preventing hepatitis B, wherein the medicament is administered by delivering to a cell of a subject having or at risk of having hepatitis B the composition.

According to some embodiments of the present invention, there is provided a composition of any one of the above embodiments for use in inactivating a hepatitis B virus in a cell.

According to some embodiments of the present invention, there is provided a composition of any one of the above embodiments for use in treating ameliorating or preventing hepatitis B.

According to embodiments of the present invention, there is provided a kit for inactivating a hepatitis B virus in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell.

According to embodiments of the present invention, there is provided a kit for treating hepatitis B in a subject, comprising an RNA molecule of any one of the embodiments presented herein, a CRISPR nuclease, and/or a tracrRNA molecule; and instructions for delivering the RNA molecule; CRISPR nuclease, and/or the tracrRNA to a cell of a subject having or at risk of having hepatitis B.

According to embodiments of the present invention, there is provided a HBV gene editing composition comprising an RNA molecule comprising a guide sequence portion having 17-50 contiguous nucleotides containing nucleotides in the sequence set forth in any one of SEQ ID NOs: 1-18936. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 to 300 nucleotides in length, 100 to 300 nucleotides in length, 150 to 300 nucleotides in length, 100 to 500 nucleotides in length, 100 to 400 nucleotides in length, 200 to 300 nucleotides in length, 100 to 200 nucleotides in length, or 150 to 250 nucleotides in length. Each possibility represents a separate embodiment.

According to some embodiments of the present invention, the composition further comprises a tracrRNA molecule.

According to some embodiments of the present invention, there is provided a method for inactivating a hepatitis B virus in a cell, the method comprising delivering to the cell a composition comprising an RNA molecule comprising a guide sequence portion having 17-50 contiguous nucleotides containing nucleotides in the sequence set forth in any one of SEQ ID NOs: 1-18936 and a CRISPR nuclease.

According to some embodiments of the present invention, there is provided a method for treating hepatitis B, the method comprising delivering to a cell of a subject having hepatitis B a composition comprising an RNA molecule comprising a guide sequence portion having 17-50 contiguous nucleotides containing nucleotides in the sequence set forth in any one of SEQ ID NOs: 1-18936 and a CRISPR nuclease.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, there is provided a method comprising removing a sequence from a HBV genomic DNA molecule (e.g. an HBV cccDNA) or a host genomic molecule which an HBV sequence has integrated into, wherein the first RNA molecule or the first and the second RNA molecules target regions flanking the sequence to be removed.

According to embodiments of the present invention, there is provided a method comprising removing a sequence from an HBV genomic DNA molecule or a host genomic molecule which an HBV sequence has integrated into. In some embodiments, the sequence is a gene or a portion thereof. In some embodiments, the sequence is a sequence encoding a protein or a portion thereof. In some embodiments, the method is for removing an entire open reading frame of HBV genomic DNA molecule or removing an entire gene of an HBV genomic DNA molecule. In some embodiments, the sequence is a non-coding sequence of HBV or a portion thereof. In some embodiments, the sequence is a regulatory element or a portion thereof. In some embodiments, the size of the excised sequence is between 10 base pairs (bp) to 100 bp, 10 bp to 3000 bp, 100 bp to 300 bp, 100 bp to 600 bp, 100 bp to 800 bp, 100 bp to 1000 bp, 250 bp to 300 bp, 250 bp to 600 bp, 250 bp to 800 bp, 250 bp to 1000 bp, or 250 bp to 3000 bp. Each possibility represents a separate embodiment.

The compositions and methods of the present disclosure may be utilized for treating, preventing, ameliorating, or slowing progression of hepatitis B.

In some embodiments, the method of inactivating a hepatitis B virus comprises delivering two RNA guide molecules to a cell to target and inactivate an HBV gene.

Any one of, or combination of, the above-mentioned strategies for inactivating a hepatitis B virus may be used in the context of the invention.

In embodiments of the present invention, an RNA guide molecule is used to direct a CRISPR nuclease to a site in an HBV sequence in a cccDNA molecule or a host genome in order to create a double-stranded break (DSB), leading to insertion or deletion of nucleotides by inducing an error-prone non-homologous end-joining (NHEJ) mechanism and formation of a frameshift mutation. The frameshift mutation may result in, for example, inactivation or knockout of an HBV gene by generation of an early stop codon and lead to generation of a truncated protein or to nonsense-mediated mRNA decay of an HBV transcript.

According to some embodiments, the present disclosure provides an RNA guide sequence (also referred to as an ‘RNA molecule’) which binds to or associates with and/or directs an RNA-guided DNA nuclease e.g., a CRISPR nuclease, to a target sequence in the HBV genome or host genome.

In some embodiments, the method comprises contacting a site in a HBV genomic DNA molecule with an RNA guide molecule and a CRISPR nuclease e.g., a Cas9 protein, wherein the RNA guide molecule and the CRISPR nuclease associate with a nucleotide sequence of the site in a HBV genomic DNA, thereby modifying or knocking-out expression of a product encoded by the HBV genome.

In some embodiments, the RNA molecule and a CRISPR nuclease is introduced to a cell harboring a hepatitis B virus. In some embodiments, the cell is in a human subject.

In some embodiments, the method is utilized for treating a subject having a disease phenotype resulting from HBV infection. In such embodiments, the method results in improvement, amelioration or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a hepatocyte or liver cell.

HBV knockout strategies include, but are not limited to, (1) truncation, for example, by targeting a sequence in a HBV genomic molecule with one guide RNA molecule to induce a frameshift or nonsense-mediated decay and (2) excision of a HBV sequence using two guide RNA molecules, for example or excision of a large portion of a HBV gene.

Truncation may be achieved by several approaches. For example, truncation may be achieved by targeting a coding sequence of an HBV genomic molecule using a single guide RNA molecule (e.g. a single guide RNA molecule or “sgRNA”). Alternatively, excision may be achieved by targeting an HBV genomic molecule with two different RNA molecules.

In some embodiments, any of the editing compositions described herein may be accompanied by small molecules that modify chromatin such as, but not limited to, methylation and deacetylation inhibitors, which can increase excision by increasing the accessibility of a DNA nuclease of the editing composition to an HBV minichromosome.

Alternatively, an editing composition comprising a nuclease (e.g. a catalytically dead CRISPR nuclease) fused to a chromatin modifier such as, but not limited to, a demethylase or a histone acetyltransferase, may increase the accessibility of the nuclease to an HBV minichromosome.

The editing composition may, for example, comprise multiple guide RNA molecules that target different sites on an HBV sequence in order to mediate excision of a regulatory element or knock-out an HBV gene from the HBV sequence.

### CRISPR Nucleases and PAM Recognition

In some embodiments, the nuclease is selected from CRISPR nucleases, or functional variants thereof. In some embodiments, the nuclease is an RNA-guided DNA nuclease. In such embodiments, the RNA sequence which guides the RNA-guided DNA nuclease (e.g., Cas9 or Cpf1) binds to and/or directs the RNA-guided DNA nuclease to a sequence within a HBV genome. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Application Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA-guided DNA nuclease of a CRISPR system, such as a Cas9 protein or modified Cas9 or homolog or ortholog of Cas9, or other RNA-guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA-guided DNA nuclease of a Type II CRISPR System, such as a Cas9 protein or modified Cas9 or homologs, orthologues, or variants of Cas9, or other RNA-guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA-guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-5-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl) carbamoyl)threonine, N-((9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

### Delivery to Cells

The compositions described herein may be delivered to a target cell by any suitable means. Compositions of the present invention may be targeted to any cell which contains HBV and/or expresses an HBV product. For example, in one embodiment the RNA molecule specifically targets a site in an HBV genome and the target cell is a hepatocyte harboring the HBV. The delivery to the cell may be performed in-vitro, ex-vivo, or in-vivo. Further, the nucleic acid compositions described herein may be delivered as one or more of DNA molecules, RNA molecules, ribonucleoproteins (RNPs), nucleic acid vectors, or any combination thereof.

In some embodiments, any one of the compositions described herein is delivered to a cell in-vivo. In some embodiments, the cell is a hepatocyte. In some embodiments, the composition is delivered to the liver of a subject. The composition may be delivered to the cell by any known in-vivo delivery method, including but not limited to, viral transduction, for example, using a lentivirus or adeno-associated virus (AAV), nanoparticle delivery, etc. Additional detailed delivery methods are described throughout this section. Non-limiting examples of suitable AAV serotypes include AAV8 hepatotropic AAV LK03.

In some embodiments, any one of the compositions described herein is delivered to a cell ex-vivo. In some embodiments, the cell is a hepatocyte. The composition may be delivered to the cell by any known ex-vivo delivery method, including but not limited to, nucleofection, electroporation, viral transduction, for example, using a lentivirus or adeno-associated virus (AAV), nanoparticle delivery, liposomes, etc. Additional detailed delivery methods are described throughout this section.

In some embodiments, an RNA molecule in the composition comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., RNA molecules within compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. The template molecule delivered for use during HDR may be, for example, adenovirus-associated vector (AAV)-based, a single-stranded donor oligonucleotide (ssODN), or a PCR-generated double-stranded DNA molecule. Such templates may be delivered by, for example, lipid nanoparticles (LNPs). Alternatively, RNA template molecules may be delivered by, for example, a lentivirus-based delivery system.

For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa®. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow; and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0)-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Application Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

The disclosed compositions and methods may also be used in the manufacture of a medicament for treating hepatitis B in a patient.

### Mechanisms of Action for HBV Knockout Methods

Without being bound by any theory or mechanism, the instant invention may be utilized to apply a CRISPR nuclease to affect a DNA break in a HBV genomic DNA molecule, such as to prevent its replication and prevent expression from the HBV genomic DNA molecule, in order to prevent or treat hepatitis B. A specific guide portion sequence may be selected from Table 1 based on the targeted HBV sequence and the type of CRISPR nuclease used (required PAM sequence). Alternatively, an HBV sequence that has integrated into a host genome may be targeted such that the CRISPR nuclease affects a DNA break on the host genome.

One strategy to knockout an HBV gene is to target an HBV sequence using one RNA molecule in order to mediate truncation or nonsense mediated decay (NMD). As a non-limiting example, a frameshift in a HBV may be introduced by utilizing one RNA molecule to target a CRISPR nuclease to a HBV coding sequence to mediate a double-strand break, which leads to generation of a frameshift mutation and expression of a truncated protein or nonsense mediated decay (NMD) of a HBV transcript.

Alternatively, an HBV gene may be knocked-out by an excision strategy that utilizes two RNA molecules. Same strategy may be implemented to excise regulatory elements that would prevent the replication of the HBV genome.

An editing composition comprising a nuclease (e.g. a catalytically dead CRISPR nuclease) fused to a chromatin modifier such as, but not limited to, a demethylase or a histone acetyltransferase, may increase the accessibility of the nuclease to an HBV minichromosome.

Alternatively, any of the editing compositions described herein may be accompanied by small molecules that modify chromatin such as, but not limited to, methylation and deacetylation inhibitors, which can increase excision by increasing the accessibility of a DNA nuclease of the editing composition to an HBV minichromosome.

One or more editing compositions comprising multiple guide RNA molecules that target multiple sites on an HBV sequence may be utilized, for example, to mediate excision of a portion of the HBV sequence (e.g. a regulatory element) or knock-out an HBV gene from the HBV sequence.

In some embodiments, the one or more editing compositions are delivered to a cell by one or more delivery vehicles. As a non-limiting example, two guide RNA molecules having different guide sequence portions and at least one CRISPR nuclease may be delivered to a cell such that a first guide RNA molecule that targets a first site is delivered by a first delivery vehicle (e.g. a first AAV particle) and a second guide RNA molecule that targets a second site is delivered by a second delivery vehicle (e.g. a second AAV particle). In another example, guide RNA molecules having different guide sequence portions are delivered to a cell by a first delivery vehicle, and at least one CRISPR nuclease, or a nucleic acid encoding the at least one CRISPR nuclease, is delivered to the cell by a second delivery vehicle. Furthermore, an first editing composition comprising a first CRISPR nuclease-RNA guide complex may be delivered to a cell by a first delivery vehicle, and a second editing composition comprising a second CRISPR nuclease-RNA guide complex may be delivered to the cell by a second delivery vehicle.

### Examples of RNA Guide Sequences Which Specifically Target HBV

Table 1 shows guide sequence portions designed to specifically target the HBV genome. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequence portions designed to
associate with specific HBV targets

| | SEQ ID NOs: | SEQ ID NOs: | SEQ ID NOs: |
|---|---|---|---|
| Target | of 20 base guides | of 21 base guides | of 22 base guides |
| HBV genome | 1-6314 | 6315-12626 | 12627-18936 |
| position 1-3182 | | | |

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: HBV Guide Sequence Portion Screening

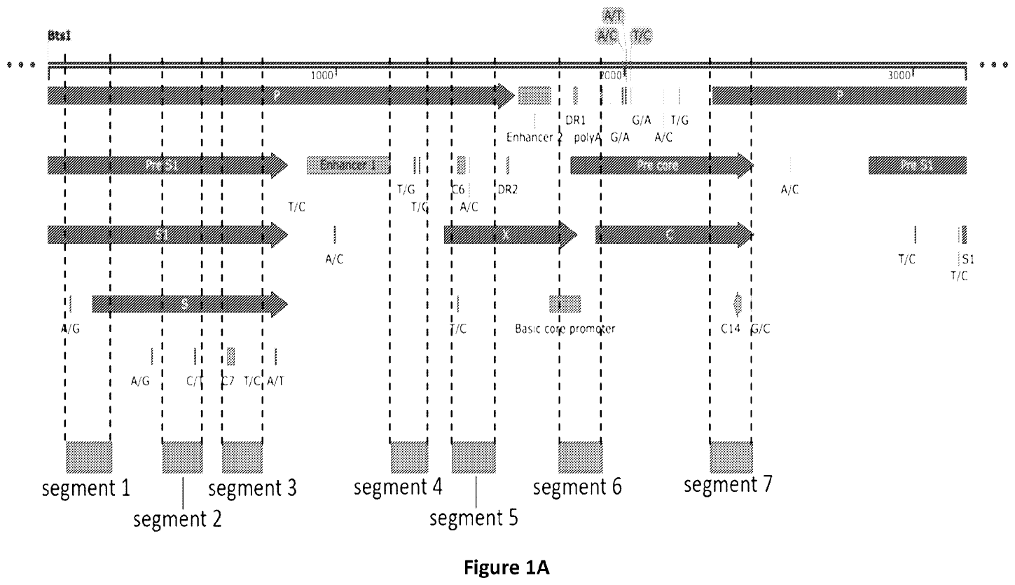

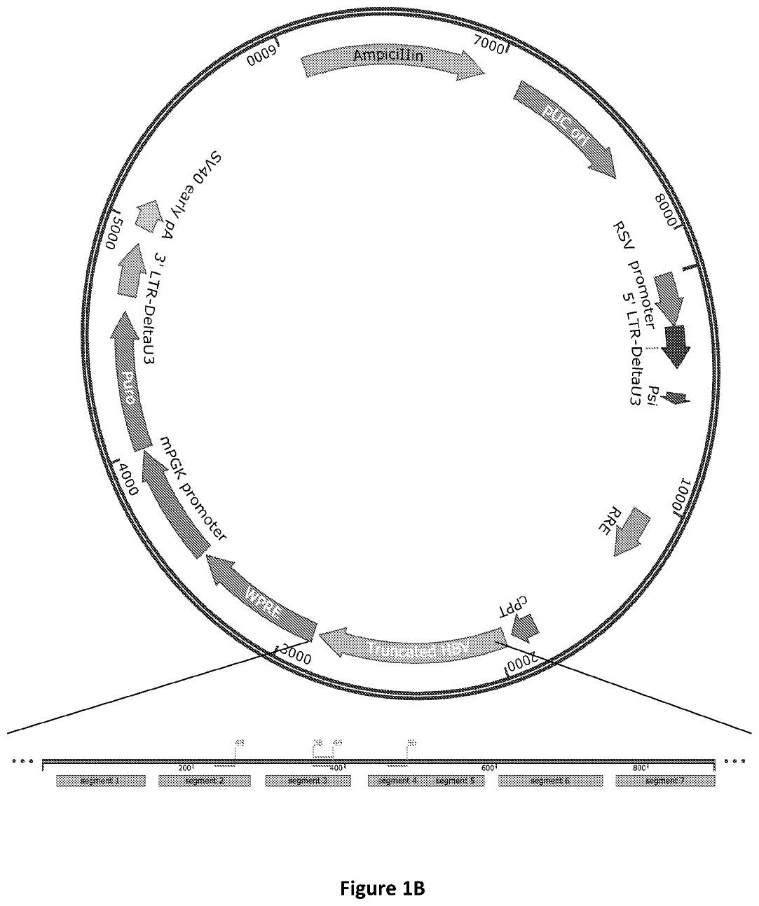

To identify optimal guide molecules for targeting conserved HBV sequences, HeLa cells were stably infected with lentiviruses harboring synthetic sequences of relevant regions of the HBV genome (FIG. 1). In hepatocyte nuclei, hepatitis B virus (HBV) genomes exist episomally in the form of covalently closed circular DNA (cccDNA). Since there is a variable copy number of HBV cccDNA molecules in each cell (typically up to 10 copies per cell), HeLa cells were infected with lentivirus harboring the synthetic sequence of HBV at three different multiplicities of infection (MOI) i.e. 2, 5, or 10 copies per cell. Stably infected cells were selected based on puromycin resistance.

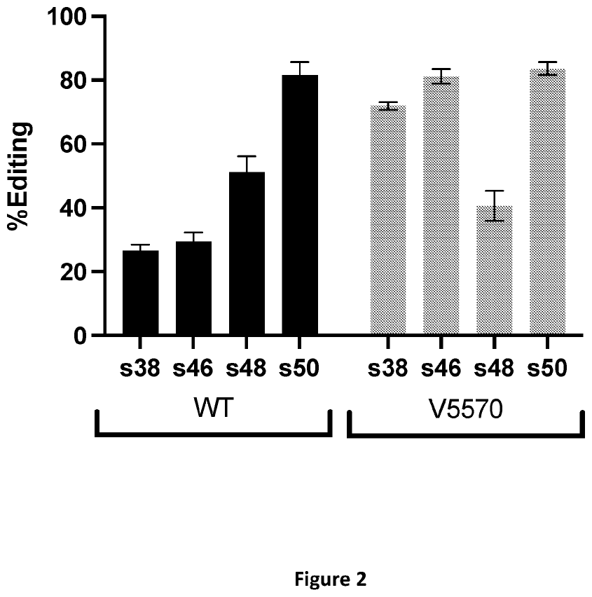

Four different guide molecules targeting distinct regions in the HBV DNA were screened for high on-target activity in HBV-infected HeLa cells (Table 2) by WT OMNI-79 nuclease and the OMNI-79 V5570 variant nuclease. Briefly, the screen was performed in 96-well format with a WT OMNI-79 or an OMNI-79 V5570 nuclease (64 ng) co-transfected with each of the guides (20 ng) using JetOPTIMUS reagent (Polyplus). Cell were harvested 72 hours post DNA transfection. Cell lysis and genomic DNA extraction was performed in Quick extract (Lucigen) and endogenous genomic regions were amplified using specific primers in order to measure on-target activity by next generation sequencing (NGS) (FIG. 2, Table 2).

TABLE 2
HBV-targeting guide sequence portions

| Target | Guide | Guide | |
|---|---|---|---|
| Location | Name | Sequence Portions | PAM |
| Segment 2 | s48 | CAGAAGAACCAACAAGAAGAUG | AGGCAT |
| | | (SEQ ID NO: 14469) | |
| Segment 3 | s38 | GCCAUUUGUUCAGUGGUUCGUA | GGGCTT |
| | | (SEQ ID NO: 16323) | |
| Segment 3 | s46 | GCCCUACGAACCACUGAACAAA | TGGCAC |
| | | (SEQ ID NO: 16342) | |
| Segment 4 | s50 | CUAGGAGUUCCGCAGUAUGGAU | CGGCAG |
| | | (SEQ ID NO: 15393) | |

TABLE 3
OMNI CRISPR Nuclease and compatible sgRNA Scaffold sequences

| OMNI | | | |
|---|---|---|---|
| CRISPR | Amino Acid Sequence | | |
| Nuclease | of the OMNI CRISPR | PAM | Compatible sgRNA |
| Name | Nuclease | Sequence | Scaffold Sequence |
| WT OMNI-79 | SEQ ID NO: 18943 | NGG | GUUGCCGCUGGAGAAAUCCAGUU |
| | | | GUUAACAAGCAGCUUGACUGCAC |
| | | | CAAAUAAGGCGGGGGCUGCGGCC |
| | | | CUCGCUUUUUU (SEQ ID NO: |
| | | | 18945) |
| OMNI-79 | SEQ ID NO: 18944 | NGG | GUUGCCGCUGGAGAAAUCCAGUU |
| V5570 Variant | | | GUUAACAAGCAGCUUGACUGCAC |
| | | | CAAAUAAGGCGGGGGCUGCGGCC |
| | | | CUCGCUUUUUU (SEQ ID NO: |
| | | | 18945) |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Agulo (2013) “Phosphoinositide 3-kinase δ gene mutation predisposes to respiratory infection and airway damage”, Science 342:866-8671.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1990) “Alpha 1-antitrypsin deficiency, emphasema, and liver disease. Genetic basis and strategies for therapy.” J. Clin. Invest. 85(5): 1343-1352.
15. Crystal (1995) “Transfer of genes to humans: early lessons and obstacles to success”, Science 270(5235):404-10.
16. Dillon (1993) “Regulation gene expression in gene therapy” Trends in Biotechnology 11(5):167-173.
17. Dranoff et al. (1997) “A phase I study of vaccination with autologous, irradiated melanoma cells engineered to secrete human granulocyte macrophage colony stimulating factor”, Hum. Gene Ther. 8(1):111-23.
18. Dunbar et al. (1995) “Retrovirally marked CD34-enriched peripheral blood and bone marrow cells contribute to long-term engraftment after autologous transplantation”, Blood 85:3048-57.
19. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
20. Gao and Huang (1995) “Cationic liposome-mediated gene transfer” Gene Ther. 2(10):710-22.
21. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
22. Han et al. (1995) “Ligand-directed retro-viral targeting of human breast cancer cells”, Proc Natl Acad Sci U.S.A. 92(21):9747-51.
23. Hu et al. (2018) “Evolved Cas9 variants with broad PAM compatibility and high DNA specificity”, Nature 556(7699):57-63.
24. Inaba et al. (1992) “Generation of large numbers of dendritic cells from mouse bone marrow cultures supplemented with granulocyte/macrophage colony-stimulating factor”, J Exp Med. 176(6):1693-702.
25. Jinek et al. (2012) “A programmable dual-RNA-guided DNA endonuclease in adaptive bacterial immunity,” Science 337(6096):816-21.
26. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
27. Judge et al. (2006) “Design of noninflammatory synthetic siRNA mediating potent gene silencing in vivo”, Mol Ther. 13(3):494-505.
28. Kohn et al. (1995) “Engraftment of gene-modified umbilical cord blood cells in neonates with adnosine deaminase deficiency”, Nature Medicine 1:1017-23.
29. Kremer and Perricaudet (1995) “Adenovirus and adeno-associated virus mediated gene transfer”, Br. Med. Bull. 51(1):31-44.
30. Macdiarmid et al. (2009) “Sequential treatment of drug-resistant tumors with targeted minicells containing siRNA or a cytotoxic drug”, Nat Biotechnol. 27(7):643-51.
31. Malech et al. (1997) “Prolonged production of NADPH oxidase-corrected granulocyes after gene therapy of chronic granulomatous disease”, PNAS 94(22):12133-38.
32. Miller et al. (1991) “Construction and properties of retrovirus packaging cells based on gibbon ape leukemia virus”, J Virol. 65(5):2220-24.
33. Miller (1992) “Human gene therapy comes of age”, Nature 357:455-60.
34. Mitani and Caskey (1993) “Delivering therapeutic genes-matching approach and application”, Trends in Biotechnology 11(5):162-66.
35. Nabel and Felgner (1993) “Direct gene transfer for immunotherapy and immunization”, Trends in Biotechnology 11(5):211-15.
36. Nishimasu et al. (2018) “Engineered CRISPR-Cas9 nuclease with expanded targeting space”, Science 361(6408):1259-1262.
37. Remy et al. (1994) “Gene Transfer with a Series of Lipphilic DNA-Binding Molecules”, Bioconjugate Chem. 5(6):647-54.
38. Sentmanat et al. (2018) “A Survey of Validation Strategies for CRISPR-Cas9 Editing”, Scientific Reports 8:888, doi:10.1038/s41598-018-19441-8.
39. Sommerfelt et al. (1990) “Localization of the receptor gene for type D simian retroviruses on human chromosome 19”, J. Virol. 64(12):6214-20.
40. Van Brunt (1988) “Molecular framing: transgenic animals as bioactors” Biotechnology 6:1149-54.
41. Vigne et al. (1995) “Third-generation adenovectors for gene therapy”, Restorative Neurology and Neuroscience 8(1,2): 35-36.
42. Walton et al. (2020) “Unconstrained genome targeting with near-PAMless engineered CRISPR-Cas9 variants”, Science 368(6488):290-296.
43. Weissman and Kariko (2015) “mRNA:Fulfilling the promise of gene therapy”, Molecular Therapy (9):1416-7.
44. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
45. Yu et al. (1994) “Progress towards gene therapy for HIV infection”, Gene Ther. 1(1):13-26.
46. Zetsche et al. (2015) “Cpf1 is a single RNA-guided endonuclease of a class 2 CRIPSR-Cas system” Cell 163(3):759-71.
47. Zuris et al. (2015) “Cationic lipid-mediated delivery of proteins enables efficient protein based genome editing in vitro and in vivo” Nat Biotechnol. 33(1):73-80.

1. A method for modifying a hepatitis B virus (HBV) sequence in a cell, the method comprising
introducing to the cell a composition comprising:
at least one CRISPR nuclease or a nucleotide sequence encoding a CRISPR nuclease; and
a first RNA molecule comprising a guide sequence portion having 17-50 nucleotides in the sequence set forth in any one of SEQ ID NOs: 1-18936 or a nucleotide sequence encoding the same,
wherein a complex of the CRISPR nuclease and the first RNA molecule affects a double strand break in the HBV sequence.

2. (canceled)

3. The method of claim 1, wherein the double-strand break is affected in a sequence of an HBV gene or portion thereof, an HBV coding sequence or portion thereof, an HBV regulatory sequence or portion thereof, and/or a conserved HBV sequence.

4. The method of claim 1, further comprising introducing to the cell a second RNA molecule comprising a guide sequence portion having 17-50 nucleotides or a nucleotide sequence encoding the same, wherein a complex of the second RNA molecule and a CRISPR nuclease affects a second double strand break in a hepatitis B virus sequence.

5. The method of claim 3, wherein the guide sequence portion of the second RNA molecule comprises 17-50 contiguous nucleotides containing nucleotides in the sequence set forth in any one of SEQ ID NOs: 1-18936 and wherein the nucleotide sequence of the guide sequence portion of the second RNA molecule is different than the nucleotide sequence of the guide sequence portion of the first RNA molecule.

6. The method of claim 3, wherein a sequence of nucleotides is excised from a molecule comprising the HBV sequence.

7. The method of claim 5, wherein the sequence of nucleotides excised from the HBV sequence comprises an HBV gene or portion thereof, an HBV coding sequence or portion thereof, an HBV regulatory sequence or portion thereof, and/or a conserved HBV sequence.

8. The method of claim 4, wherein the first or second RNA molecule comprises a guide sequence portion that targets a sequence that is located up to 500 base pairs from an HBV gene, an HBV coding sequence, an HBV regulatory sequence, and/or a conserved HBV sequence that is to be excised by the first and second RNA molecules.

9. The method of claim 1, wherein the cell is a liver cell or a hepatocyte.

10. The method of claim 8, wherein the cell is from a human subject.

11. The method of claim 9, wherein the human subject suffers from chronic or acute hepatitis B.

12. The method of claim 1, wherein the HBV sequence is located on an HBV covalently closed circular DNA (cccDNA) molecule.

13. The method of any one of claims 1, wherein the HBV sequence is located on a genomic DNA molecule that an HBV sequence has integrated into.

14. A composition comprising:
at least one CRISPR nuclease or a nucleotide sequence encoding a CRISPR nuclease; and
a first RNA molecule comprising a guide sequence portion having 17-50 nucleotides in the sequence set forth in any one of SEQ ID NOs: 1-18936 or a nucleotide sequence encoding the same,
wherein a complex of the CRISPR nuclease and the first RNA molecule affects a double strand break in an HBV sequence in a cell.

15. (canceled)

16. The composition of claim 13, further comprising a second RNA molecule comprising a guide sequence portion having 17-50 nucleotides or a nucleotide sequence encoding the same, wherein a complex of the second RNA molecule and a CRISPR nuclease affects a second double strand break in a hepatitis B virus sequence.

17. The composition of claim 14, wherein the guide sequence portion of the second RNA molecule comprises 17-50 contiguous nucleotides containing nucleotides in the sequence set forth in any one of SEQ ID NOs: 1-18936, and wherein the nucleotide sequence of the guide sequence portion of the second RNA molecule is a different nucleotide sequence from the nucleotide sequence of the guide sequence portion of the first RNA molecule.

18. A method for treating hepatitis B, the method comprising delivering to the cell the composition of claim 13.

19. A method for treating, ameliorating the symptoms of, or preventing hepatitis B, the method comprising delivering to a cell of a subject having or at risk of having hepatitis B the composition of claim 13.

20. (canceled)

21. A medicament comprising the composition of claim 13 for use in inactivating a hepatitis B virus in a cell, wherein the medicament is administered by delivering to the cell the composition of claim 13.

22. (canceled)

23. A medicament comprising the composition of claim 14 for use in treating, ameliorating the symptoms of, or preventing hepatitis B, wherein the medicament is administered by delivering to a cell of a subject having or at risk of having hepatitis B the composition of claims 14.

24.-25. (canceled)
